SEQUENCE LISTING (24 sequences)

SEQ ID NO: 1 - DNA, 10798 bp, Lycopersicon esculentum
gatctcgataagttatgtcttgttggaatcgatatcaaataaccgtcgacggtatctttgatatgaggtagcgctcaatgatataaattgtgatgaggatcttgaattcaaatctgtcatatagtgtgaacagataaatggttagccaagtaaaatgcacaattcaagtatattttgtttcacttagaaaagtgacattttggactggtagtccataaatcaaggtataatgtcagtggggtacaaataaattattatgtgatagtataaccgtaagatatcaaatacggtttgtgccttggggcataaaggtttatcgcaaaaatcctgacattattggagatgttttctcctttggtggatgcaatgaggtttgttttgatctggcaacatatgaaaaacttgaatgcatgtaatgaaaaattgtaatgaaggttatatgaaaatccttgaaacaatccaggtgtctgaagcatataaaggttgaaagaaacttatccaataaagcttcaagaatccttatatggattgaaatagtcaaggaagaaaaagggtacaaaagaatgaccctaattgtccttgtatttttatgaaaaggtcttggtaagacaaaattttgtcttgacctacagattgttaatttgacaaataaaatatttgtctaacagacaacagtgcacatacactgaaaaattttgatgcaattttatgtggatatatcgcattcattgagtaccccaatgattatgagatcacttgacataaatgatgattcagtttgatctcaaaagaaggataagagtttcttggtgatgaaactctatcttggtgcaatgagggcactagtgcatcttactaacaatatttgactagatatttgttttgcagtaaatttactggcaagattcagtttctccccgataaaaggacattgaaatggtgttgagcacatgaatgaatatcctcaaaggaccatagttatgggtttattctatcccgaggaatccaagacaaaattgattgattacgcagatgcagaatatttatctgatccgcataaagctctatctcaagcacgctatgtgtttgcatgtggaggcacaataatatcctggggatcaatgaagcaaatgttgctctgcagaaataaaagtcctccatgaagcaagtcaaaagtgcgtctggttgagataaatgacacaccatattcaagaaatgtgtggtttttctttaaaaaaagaatataccaaccacaatgtacaaagattggagacatcatcacaagaaatcaagtgatgttttaatcagggggagtacaatacgcgttgcactctttttcccttgatcgaggtttttttcccactggattttcctgacaaggtttttaatgaggcaacaaatggtgcgtatcaaaagatatgtgtactctttttccttcactagaattttttcccacagggtttttcctagtaaggttttaacgaggcacattatctatggacatccaagggggagtgttataaatacattgaattaagtggatagtccataaggttggcacatgaacaaccattcatattcactaggtgacatgaacctttttggataagaatgtatctatttattatgatacttaatatggtaatctttggagtgatttctcactctataaatagagttgttcattcactattgtaatatatacatatgagacttgaatacacttgaatacgaagaaagtcttatcttccatcttacttctcttgtcttctctctttatgattatattcttatgagcttgattttataacacgaatctcattatacgaaaagttttactatttatatttaattaatagaggatttaaactttttaaatttctgtctttatagatgagaacttgtctttttgttgaatccaactaaacattcaatgaagacaaatcaacctgtaaatccctttcaagtaggatttattcgaatctcattatacgaaaagttttactatttatatttaattaatagaggatttaaactttttaaatttctgtctttatagatgagaacttgtctttttgttgaatccaactaaacattcaatgaagacaaatcaacctgtaaatccctttcaagtaggatttattcgaatctcattatacgaaaagttttactatttatatttaattaatagagaatttaaactttttaaatttctgtctttatagatgagaacttgtctttttgttgaatccaactaaacattcaatgaatacaaatcaacctgtaaatccctttcaagtaggatttattcgaatctcattatacgaaaagttttactatttatatttaattaatagagaatttaaactttttaaatttctgtctttatagatgagaacttgtctttttgttgaatccaactaaacattcaatgaatacaaatcaacctgtaaatccctttcaagtaggatttattcgaatctcattatacgaaaagttttactatttatatttaattaatagagaatttaaactttttaaatttctgtctttatagatgagaacttgtctttttgttgaatccaactaaacattcaatgaatacaaatcaacctgtaaatccctttcaagtaggatttattcgaatctcattatacgaaaagttttactatttatatttaattaatagagaatttaaactttttaaatttctgtctttatagatgagaacttgtctttttgttgaatccaactaaacattcaatgaatacaaatcaacctgtaaatccctttcaagtaggatttattcgaatctcattatacgaaaagttttactagttatatttaattaatattcaagtctcaatttttttttaaatatttacattccacattttaatctataatgaaagttactaaaatatactatcaaggagaaaatatacaaaatggcccataacgatagtctttaatatataataaatatgttcatttggatccttaatatatttcacttgattaaaataataataaatgtataataaaaagtggtcattttggtcttttgtcctaaacatagagtttttttaccttcaaagaaaaatcttccataaaatctaatactatttttttttaatttctccaacaaaatttattattttctcttttaaatattattttactgacctaataacagtttttattttgagcaagaaaagtagtaaattttgttaaataaagaaccaaaataaatcattttaatcaaagtaaaatataataacgattaaaataaagtatacattaagtcatttcaatgaagtgaaataaatgaagaagtaaaataaaaaaattaaccaaacagtaagcatagttttggtcattttctctaatcccaagtgtacctcaaattataaaagtccttttgttactcaatttcgttggtcccagtcattttctgtgttcatcacctatatatatagcagtagactagtagcttctcccattcctctatcttctattatggccactcagtgttatgaccccgaaaactccgcctctcgttacacattactcccggatcaacccgattccggccaccggaagtcccttaaaatcatctccggcattttcctctccgttttccttttgctttctgtagccttctttccgatcctcaacaaccagtcaccggacttgcaaatcgactcccgttcgccggcgccgccgtcaagaggtgtttctcagggagtctccgataaaacttttcgagatgtagccggtgctagtcacgtttcttatgcgtggtccaatgctatgcttagctggcaaagaacggcttaccattttcaacctcaaaaaaattggatgaacggtaattaactttcttattttgacttttctttaatttcttttttatttgatcttaaaattgaaattatttataaatacttataacagttcttttttttctcaatgatatttatggctattgatctgttgggggtatcttttggattctgattggatgctattctgcagatcctaatggtgagttcaaagttaattattatcactattttctgctagtttttaattaattatattcttaaactatgattataacttttaaagcaatctcatgaatgagcaaatcattaattcgggtgcttatgtatatcatctcggttaatccttttaccttatactcaaaaacaaatattactcccttcaaaataattgatgtttgacataatcaatgtgatgtttaatttttttttctttcaaatttgcccttcctaacccctataatgattatgtcaaatccaaagtgaaaagactatcataattacatatgctttagtcacaattaattcatgttaaatcatcaatagttttggattggagggagtactcattaggaaaaataattaagctaaatcattcttattttcactgtacattatttagattaagggtgaaataggggaggaatcaattatcttatttttctaaatggacaagtattttgaaataacaaattttaagaaaacacgtcaagtcaaatagagtaggatggatggagtaaattctaacctttctagatattcataaaaattagttgaacagacattttaataaagaccacaagttgatgaattaagcttgttgttccaatataattgggattaacatgagatcttgtggcagtaatgttttttgcttttgtgcaattttccaataaaaagaaaacacttgattgggtcagtattatacaagtttggaaaccaatcacgttatgtgggtcatacttttttgtagtaatgtaataataccaatagtggggcccccactcaaagtaatccatcttccacttgatttttttatttttttttgaaatggagtaggttatcttggccgcttagcaattactattatcatgagtaaatgacggaaattataaatttttaagataaaattattattaatcttttataattttatggttataaaagtctctcaaactaatacaataatataagcgctgatacatgagtctgatgtgcgagatacattaatctgataggtaaaaatgaggaactagaaatttataaaactaatatgaataatgataataagataacttaaatgtgaaatttctatcatttctcctaacataccactagtgaaatttgtttacgtatcttgttgaagaaaatcttatccaaaagtcaaaaataaaaactcgtggccaaattttcaaaaaaaaaagaaggttatctttttgccgcaaaaagcatagcaattttggtacggaacgtattgagattttgtagagtattttataattcaaattgcatagaaaagtcttacctatacaagtaaaaactttgaaatttctattaacgtgaataaattggttaacaggaccattgtatcacaagggatggtaccaccttttttatcaatacaatccagattcagctatttggggaaatatcacatggggccatgctgtatccaaggacttgatccactggctctacttgccttttgccatggttcctgatcaatggtatgatattaacggtgtctggacagggtccgctaccatcctacccgatggtcagatcatgatgctttataccggtgacactgatgattatgtgcaagtgcaaaatcttgcgtaccccgccaacttatctgatcctctccttctagactgggtcaagttcaaaggcaacccggttctggttcctccacccggcattggtgtcaaggactttagagacccgactactgcttggaccggaccacaaaatgggcaatggctgttaacaatcgggtctaagattggtaaaacgggtgttgcacttgtttatgaaacttccaacttcacaagctttaagctattggatggagtgctgcatgcggttccgggtacgggtatgtgggagtgtgtggacttttacccggtatctactaaaaaaacaaacgggttggacacatcatataacgggccgggtgtaaagcatgtgttaaaagcaagtttagatgacaataagcaagatcattatgctattggtacgtatgacttgggaaagaacaaatggacacccgataacccggaattggattgtggaattgggttgagactagactatgggaaatattatgcatcaaagactttttatgacccgaagaaagaacgaagagtactgtggggatggattggggaaactgacagtgaatctgctgacctgcagaagggatgggcatctgtacaggtatggacttggatgaacacattgttttgttattttactttgcaccatacacagcgtctagttgtatcgtaataatcatggtagggaaatttcttatttagagaaagttgttataatcaatgcatttgtaggtgaagtaaattctgaattgtatatgaaacgtgtctaatagtgtttcgaaataacagagtattccaaggacagtgctttacgacaagaagacagggacacatctacttcagtggccagtggaagaaattgaaagcttaagagtgggtgatcctactgttaagcaagtcgatcttcaaccaggctcaattgagctactccgtgttgactcagctgcagaggtttgttgcgttacttttgttttaaattacaaacacgcgcttaatctgcagtcccaaaacttgtttagctattgtgcagttggatatagaagcctcatttgaagtggacaaagtcgcgcttcagggaataattgaagcagatcatgtaggtttcagttgctctactagtggaggtgctgctagcagaggcattttgggaccatttggtgtcatagtaattgctgatcaaacgctatctgagctaacgccagtttacttttacatttctaaaggagctgatggtcgtgcagagactcacttctgtgctgatcaaactaggtttgcttttctatctggcacaattaatttgtccttgtaaaatggagatggataaaagtagcgggttgttgatctgatatatgcagatcctctgaggctccgggagttggtaaacaagtttatggtagttcagtacctgtgttggacggtgaaaaacattcaatgagattattggtaagtgataatgattcccttattttaccttgattttattccatttcttcacttcacaataattaaagtacttggcagttgcatttgagtaaaaggttttttataaactgaattttaggtggatcactcaattgtggagagctttgctcaaggaggaagaacagtcataacatcgcgaatttacccaacaaaggcagtaaatggagcagcacgactctttgttttcaacaatgccacaggggctagcgttactgcctccgtcaagatttggtcacttgagtcagctaatattcaatccttccctttgcaagacttgtaatcttctttatttcgttttttttttctttttcatttgaaggttatttcaccgacgtcccatcaagaaagggaagagggagatcaatatatgtagtgttattcgccctaccttaggattagatgtcatctagcaatgtcaaatctagtagagtatacaatgtatgggttcctggaaaccgagtagagcttacctggattctatgtaaactaagaaagctcagcaaatatatgcacaaataatttacagaaacaacttgggaatgttgacaaacttgattattttttcttttatataactagtaataacggcaagctctccgcaatctcgttgagcaaaagtataaatggttacgagccacctaaatatttttgttcaacgagattggaattggagcttattatacacaacatatacaacaatgattcatcttctaactcatacaattctatacgtaaggtcgaagttaggagggagtgagcaacttggtaaaaagtatatggtataagtaagatatttttaaatgtattatgtatcagttgtactcaatcaaagagcggataaatacaattgatacaatatacaaaatagttatgcactaaataataaatagaggataaaatgtaaaagaaatacaaaatataattctctcgatctcgctcccgtctctcctctctcgatctcactcatctctcttctcttaatatgtattcattttaatacaaattagtttctatttgtattttttcttcaaaattcacgaaaaaaaatatatataaatataaatgcatagcgaacaagaatattattatgaatcataaataatgaaactgtagttatggaatacttttaagggttaatgtttgttgtttttgaaatttcccctcttgaagcccttaagtgcaaatcttgaatccactatgaatatgattcattctttatacatatacaataataatgatacatttctatttacgaatgatataattcccgtacaaataaatttagagttacaaaagaagatcagcccagcccatctaattcaagcctcgtgggccaagaaatttaatgagctaaggaaggttggccctttatttgaaagtgcctaaattgttcaactcaacctaattttagaagggccacaaactgggggggttagcatttttttcctttttaaacttaaagctctataccatcaagtaaatgagactattttcaaatcaaatatggtaacaatggtgttttttcaataacactaacaaaaaatttgtatgattaacatgtaccttggatactacatgcccaagctacatgtatatgttgtgatgcattccaaatatgcaagcgagataagagcgaccaagatgggtgggaggcgagggcttggaatttgtttatatatcctagatacatgcgaatccatttgaatgaagtccttctagaataaatagacgtatcgaaatgcaccaaaatctagtaagatttgtaatgttacagcataacgtgcatctaagtaattagctagctcatacactagtgagatccttttagttaccgtatataaatagttttgacccatgggacgatcctaacctgttcccgatcaagactcaagggcttataagtcctaatgttgaatggtcttgtaaatcctatcacaaccataccccaataccgagttgggttggaccggctccatgggcttagcaaactttgacatatctacacataatggaacaaatgaaaaaaaaaatacgaaatgaaattatttttaaaacaataaagacaatatttttttagagaaagttacaaaattatatacaacttaatattattatatcctctaaaaattcctatctttgaattaaatacaaaaatttcctttttccttctctctcttttttcatccggatacatcactcgacctctatgaaatacaccacaattttgtttgtgtatactaatatggtagaaatattattaccgatacataaccccaattatttcaaatataattatattagtgatacacaacttatttattgtttgttatatatatagagcgaatgagcaatgtatccacaagttttgaaaaatccaaaatcatttatttaaaaaacttttaagataatgtgtaattaacgcctaaaaactattgaggtttctgtattctgtattgtattccttttaaggaaaaatatataataacaaactattaattcaaattaaatgttatatacacaatttgatttaacctgtagcaaaatattttcattcgcctctctccctaggtttctcactcgccactctcgcttttatacaaacacaaatgtataaaatgtgtttgtgtttgtataaagcgagagaaaatgtatatacaaatatgaatacatatattttcgtcctatatacttataatgatacaaatacagatcttttcctatccagttctcttttgtctttctcactttatacaaacacaaattatacaaattacaatgtataattattgttgcataaagcgagagagagattcgatatacaaatagtttatttcgattcaattatatataaattcaaattttatgcagatatgcaaacaaataaaataaaatttgagaggctgtcagcgatttatgccaacgatttatacaaatgacctaccaccgaaattatacaaatctgaagcattgccagcgagctatacaatctgatgctccataacaaacataaaatttatcatggaacgtaaatatacaaactatgactataacattcaaatataatttttatgtttgccatatatgaaaattgatctaagcctttcgaactatccgatgtcaatagtttcacccagatagccattaatatcaaagttcaggcccagatcattgggataatttgggcctatattgtggaccgtgactcgaaaaacacctaatgctacaggctacaccaaattgattaatgatttctcatcttctgaaaacaaaataaatttataatttttatattacataaatatttttttcccgctaaattcaaagtagtcaaacattcaaaaatatttaaactgataatcagagctcaagtcaccttttcatttatactattattatatttttttaatattagagacaaaaaagaaaagctctcatattaaataataaaatatatagaattgacagaaccatttgaccattcttctcatagttaaaatagtatataattgggctcgactttatataaaattctgatatattatttaatattcttctttgcttttccttttctgcattactttttttttccatttaaataataatacaggtttatgggtattataaaacggatcc

SEQ ID NO: 2 - DNA, 3616 bp, Lycopersicon esculentum
atggaattatttatgaaaaactcttctctttggggtttaaaattttatttattttgcttatttataattttatcaaacattaatagggcatttgcttctcataatatttttttggacttgcaatcttcaagtgctattagtgtcaagaatgttcatagaactcgttttcattttcaacctcctaaacattggattaatggtatgttcattttttttttattttatataacatgcgataaatttaacgttagcaatgtggtttgttatttaaattcgaatttgattatatgactttgcttatataaatatacatagtaataaaagtttgtgtataaatgcatgtcatatacatttattgacttggtatatatatcagtacgattaaattaattgatggtgcaattaatattgcattatttaggtgataaaactacagaaattaacgaaaatattttttttatatagagaagttcaaatgttgagggttcttttatggttacattggtttaaaatgttttttgttaactatctttatagctacatatatataagagtgatcattctttatatttcaaaattatatctacatacacacatatacatcattatgtggttcatttatggtagttttcagtattcgatatttatttttaagtttaatttatttaaatctgcgttaaaatatctcactttgaaagatagaatcactcctgaccaactatgagtaactcgattctcaaaatttaaattcggaattagattaattatcatggcaagagaactaccacgttttggataagaatgtgcaaaagagagaaagaaacatgaaatatataaaaacctaagattttggccatggaaagttaggtgcgaattaatttgttgaaggcaccctttattattattattataattattattattattaatgaaatatagtgacatttcatactcatatattgtgtgcatttaattaatatatgtaggtcttatgttaatttaaacttaccaaacatattgtctcttataaagttgactccccccctcaaccgccaaccccacccccacccccaccccacccaaaaaaaatacctcatcaatttcggtttttatatgactcaattttcttgtttaatttgttatctacagaacggactactttctatatcattctacataatatgtatattttttataatccaataaatctcatgacacgttttcagatcataattttgcaaacacctttttctttattttttaattaggtatatcacataaattaaaaggattcattaattttcgcagagaaaactaattagtttctgtgtttttcacctttcatttattaattactacataatttttaatcaataattgatgaaagactatgtaatgtattctattatcttcactaatcattttttttttgtataattcttatatggtctctctccattggatgcctttcaaatatacaaagaccctaatggtaagttagattatttttcatttaattttatcaataactcaatgatattattgattttcattttatttttcaaacagcaccaatgtattataatggagtgtatcatttattctatcaatacaatccaaaaggatcagtatggggcaatattatttgggctcattcagtctcaaaagacttgataaattggatccatttagaacctgcaatttatccatccaaaaaatttgacaagtatggtacttggtctggatcatcaactattttacctaataacaaacctgttatcatatacaccggagtagtagattcgtataataatcaagtccagaactacgccatcccggctaacctatctgatccatttcttcgtaaatggatcaaacctaacaacaacccgttgatcgtccctgataacagtatcaatagaactgagtttcgcgatccaactacagcttggatgggccaagatgggctttggaggattttaatagcaagtatgagaaaacatagagggatggcattgttgtatagaagtagagattttatgaaatggatcaaagcccaacatccacttcattcatctactaatactggaaattgggagtgtcctgattttttccctgtattatttaatagtaccaatggtttagatgtatcgtatcgcggaaaaaatgttaaatatgtcctcaagaatagtcttgatgttgctaggtttgattattacactattggcatgtatcacaccaaaatagataggtatattccgaataacaattcaattgatggttggaagggattgagaatcgactatggtaatttctatgcatcgaagacattctatgatcctagcagaaatcgaagggttatttggggttggtcaaatgaatccgatgtattacctgacgatgaaattaagaaaggatgggctggaattcaaggtattccgcgacaagtatggctaaaccttagtggtaaacaattacttcaatggcctattgaagaattagaaaccctaaggaagcaaaaggtccaattgaacaacaagaagttgagcaagggagaaatgtttgaagttaaagggatctcagcatcacaggtttcaacttttccttattaaactatagtcttttaaatatcattaatctacttcttatatgtataatcaatgtataactattatatcaaatgcacatgatcgattgattatacatttgctatatatatatctctattatatcaattgcactgtctcatcttgcatttctttgatcgtaggctgatgttgaagtgctgttctcattttcaagtttgaacgaggccgaacaatttgatcctagatgggctgacctatatgcccaagacgtttgtgccattaagggttcgactatccaaggtgggcttggaccatttgggcttgtgacattagcttctaaaaacttagaagaatacacacctgttttcttccgagtgttcaaggctcaaaaaagttataagattctcatgtgctcagatgctagaaggtttgtttcttcaatccaattaattgtaatgatcgaagttcacatcttctccaaattgagtaaatcgagaattataatgacccgactttgatatcatgataagaaatgcatttacttatagatcgcccgttagtgtcattaaaaaactctaaccttgtttaggttttttttttttttaattaatgagcagatcttccatgagacaaaatgaagcaatgtacaagccctcatttgctggatatgtagatgtagatttagaagacatgaagaagttatctcttaggagtttggtaagttttgctttcacaatttttatttatttataatttatttgatcaaaactttcaagattcgattaatttgaagagtaacgatttgtgtttgactaatcaatttgtatcatatgcatatttttttttagattgataactcagtagtggaaagtttcggtgctggtggcaaaacatgcataacatcaagggtgtatccaactttagcgatttatgataatgcacatttatttgtttttaacaatggctctgagacaatcacaattgagactctgaatgcttggagcatggatgcatgtaagatgaactaa

SEQ ID NO: 3 - DNA, 26 bp, Artificial
  LeTiv1L2 primer
cagtgttatgaccccgaaaactccgc

SEQ ID NO: 4 - DNA, 29 bp, Artificial
  LeTiv1R2 primer
tgaggttgaaaatggtaagccgttctttg

SEQ ID NO: 5 - DNA, 27 bp, Artificial
  LeTiv1L8 primer
ggccgggtgtaaagcatgtgttaaaag

SEQ ID NO: 6 - DNA, 27 bp, Artificial
  LeTiv1R8 primer
ttgagcctggttgaagatcgacttgct

SEQ ID NO: 7 - DNA, 23 bp, Artificial
  LeTiv1L4 primer
cagaggcattttgggaccatttg

SEQ ID NO: 8 - DNA, 24 bp, Artificial
  LeTiv1R4 primer
gagtcgtgctgctccatttactgc

SEQ ID NO: 9 - DNA, 27 bp, Artificial
  LeTiv1L6 primer
gcccccactcaaagtaatccatcttcc

SEQ ID NO: 10 - DNA, 28 bp, Artificial
  LeTiv1R6 primer
taccattgatcaggaaccatggcaaaag

SEQ ID NO: 11 - DNA, 27 bp, Artificial
  LeTiv1L9 primer
ggacaaagtcgcgcttcagggaataat

SEQ ID NO: 12 - DNA, 27 bp, Artificial
  LeTiv1R9 primer
agtcgtgctgctccatttactgccttt

SEQ ID NO: 13 - DNA, 25 bp, Artificial
  LeTiv1L10 primer
tcgttggtcccagtcattttctgtg

SEQ ID NO: 14 - DNA, 27 bp, Artificial
  LeTiv1R10 primer
tgcagaatagcatccaatcagaatcca

SEQ ID NO: 15 - DNA, 26 bp, Artificial
  LeTiv1L11 primer
caggaccattgtatcacaagggatgg

SEQ ID NO: 16 - DNA, 24 bp, Artificial
  LeTiv1R11 primer
tgctttacacccggcccgttatat

SEQ ID NO: 17 - DNA, 28 bp, Artificial
  Lin5L2 primer
tggtcaaatgaatccgatgtattacctg

SEQ ID NO: 18 - DNA, 20 bp, Artificial
  Lin5R2 primer
ccaaatggtccaagcccacc

SEQ ID NO: 19 - DNA, 27 bp, Artificial
  Lin5L4 primer
ccatcccggctaacctatctgatccat

SEQ ID NO: 20 - DNA, 27 bp, Artificial
  Lin5R4 primer
tgttgttcaattggaccttttgcttcc

SEQ ID NO: 21 - DNA, 26 bp, Artificial
  Lin5L3 primer
cacctgttttcttccgagtgttcaag

SEQ ID NO: 22 - DNA, 20 bp, Artificial
  Lin5R3 primer
atgttttgccaccagcaccg

SEQ ID NO: 23 - protein, 636 aa, Lycopersicon esculentum
MATQCYDPENSASRYTLLPDQPDSGHRKSLKIISGIFLSVFLLLSVAFFPILNNQSPDLQIDSRSPAPPSRGVSQGVSDKTFRDVAGASHVSYAWSNAMLSWQRTAYHFQPQKNWMNDPNGPLYHKGWYHLFYQYNPDSAIWGNITWGHAVSKDLIHWLYLPFAMVPDQWYDINGVWTGSATILPDGQIMMLYTGDTDDYVQVQNLAYPANLSDPLLLDWVKFKGNPVLVPPPGIGVKDFRDPTTAWTGPQNGQWLLTIGSKIGKTGVALVYETSNFTSFKLLDGVLHAVPGTGMWECVDFYPVSTKKTNGLDTSYNGPGVKHVLKASLDDNKQDHYAIGTYDLGKNKWTPDNPELDCGIGLRLDYGKYYASKTFYDPKKERRVLWGWIGETDSESADLQKGWASVQSIPRTVLYDKKTGTHLLQWPVEEIESLRVGDPTVKQVDLQPGSIELLRVDSAAELDIEASFEVDKVALQGIIEADHVGFSCSTSGGAASRGILGPFGVIVIADQTLSELTPVYFYISKGADGRAETHFCADQTRSSEAPGVGKQVYGSSVPVLDGEKHSMRLLVDHSIVESFAQGGRTVITSRIYPTKAVNGAARLFVFNNATGASVTASVKIWSLESANIQSFPLQDL

SEQ ID NO: 24 - protein, 584 aa, Lycopersicon esculentum
MELFMKNSSLWGLKFYLFCLFIILSNINRAFASHNIFLDLQSSSAISVKNVHRTRFHFQPPKHWINDPNAPMYYNGVYHLFYQYNPKGSVWGNIIWAHSVSKDLINWIHLEPAIYPSKKFDKYGTWSGSSTILPNNKPVIIYTGVVDSYNNQVQNYAIPANLSDPFLRKWIKPNNNPLIVPDNSINRTEFRDPTTAWMGQDGLWRILIASMRKHRGMALLYRSRDFMKWIKAQHPLHSSTNTGNWECPDFFPVLFNSTNGLDVSYRGKNVKYVLKNSLDVARFDYYTIGMYHTKIDRYIPNNNSIDGWKGLRIDYGNFYASKTFYDPSRNRRVIWGWSNESDVLPDDEIKKGWAGIQGIPRQVWLNLSGKQLLQWPIEELETLRKQKVQLNNKKLSKGEMFEVKGISASQADVEVLFSFSSLNEAEQFDPRWADLYAQDVCAIKGSTIQGGLGPFGLVTLASKNLEEYTPVFFRVFKAQKSYKILMCSDARRSSMRQNEAMYKPSFAGYVDVDLEDMKKLSLRSLIDNSVVESFGAGGKTCITSRVYPTLAIYDNAHLFVFNNGSETITIETLNAWSMDACKMN

(19) United States
(12) Patent Application Publication
(10) Pub. No.: US 2005/0120418 A1
(43) Pub. Date: Jun. 2, 2005

(54) Tomatoes having altered acid invertase activity due to non-transgenic alterations in acid invertase genes

(75) Inventors: Sal Fuerstenberg, Woodland, CA (US); Ann J. Slade, Kenmore, WA (US)

Correspondence Address: Alison J. Baldwin;McDonnell Boehnen Hulbert & Berghoff, 32nd Floor, 300 S. Wacker Drive, Chicago, IL, 60606, US

(73) Assignee: Anawah Inc.

(21) Appl. No.: 10/998,232

(22) Filed: Nov. 5, 2004

Related U.S. Application Data
(60) Provisional application No. 60/518,112, filed on Nov. 6, 2003.
(60) Provisional application No. 60/520,811, filed on Nov. 17, 2003.
(60) Provisional application No. 60/547,720, filed on Feb. 25, 2004.
(60) Provisional application No. 60/559,998, filed on Apr. 5, 2004.

(51) Int. Cl. A01H 1/00; C12N 15/82; C12Q 1/68; A01H 5/00

(52) U.S. Cl. 800/287; 800/317.4; 435/6

(57) ABSTRACT

A series of independent non-transgenic mutations found in acid invertase genes of tomatoes; tomato plants having these mutations in their acid invertase genes; and a method of creating and finding similar and/or additional mutations in acid invertase genes by screening pooled and/or individual tomato plants. The tomato plants of the present invention exhibit altered acid invertase enzyme activity and altered concentrations of sugar in the tomato fruit without having the inclusion of foreign nucleic acids in their genomes.

### FIELD OF THE INVENTION

This invention concerns non-transgenic mutations in acid invertase genes of tomato and tomato plants having these non-transgenic alterations in their acid invertase genes. This invention further concerns tomato plants having altered sugar accumulation in their fruits due to non-transgenic mutations in at least one of their acid invertase genes. The invention further concerns methods that utilize non-transgenic means to create tomato plants having mutations in their acid invertase genes.

### BACKGROUND

A major objective of tomato breeders has been to develop fruit with high sugar content for improved flavor and quality of fresh and processed tomatoes. Factors that regulate sugar content in tomatoes appear to be complex. However, recent studies have utilized genetic and biochemical techniques to study traits that may be responsible for the naturally occurring differences between sugar accumulating wild tomato species, such as Lycopersicon pennellii, Lycopersicon chmielewskii, and Lycopersicon hirsutum, and the cultivated tomato, Lycopersicon esculentum. Because sugar content can represent up to 15% of the fruit's wet weight in wild tomatoes compared to only 5% in cultivated tomatoes, researchers and breeders hope to better understand those traits responsible for sugar accumulation so that they may be introduced into cultivated varieties. By identifying and modifying specific genes, it may be possible to utilize the sugar accumulating trait commercially but avoid other undesirable quality and agronomic characteristics of wild tomatoes.

Species specific differences in the enzyme acid invertase may be responsible for the differences in sugar accumulation between cultivated and wild tomatoes. Invertases are enzymes that irreversibly cleave sucrose into glucose and fructose. Multiple invertase enzymes (acid invertases as well as neutral and alkaline invertases) have been identified and classified according to their optimal pH range for cleavage. Two major acid invertase activities have been identified. Intracellular soluble acid invertase activity (also known as vacuolar invertase activity) is located in the vacuole and is encoded by at least two genes, TIV1 and TIV2. Extracellular insoluble acid invertase activity (also known as cell wall or apoplastic invertase activity) is bound to the cell wall and is encoded by at least four genes, Lin5, Lin6, Lin7, and Lin8.

The various roles of the different invertases are not completely understood. Invertase, along with the enzyme sucrose synthase, plays an important role in carbohydrate partitioning in plants by establishing the sucrose concentration gradient that drives sucrose transport between source tissues (leaves) and sink tissues (fruit, seeds, tubers, shoots, and roots). Invertase is also believed to regulate the entry of sucrose into the various utilization pathways. By controlling the relative levels of sucrose versus glucose and fructose, invertase is postulated to play a role in multiple processes including growth and differentiation (e.g., stature, fruit set, fruit and tuber size) and the response to stress (e.g., wounding, fungal infection, starvation, and temperature).

It is not clear which acid invertase is responsible for the species specific differences in sugar accumulation and both TIV1 and Lin5 have been implicated. Whereas cultivated tomatoes stop accumulating sugar once the fruit has reached full size, wild tomatoes continue to accumulate sugar until late in development. The wild tomato Lycopersicon chmielewskii has lower acid invertase activity levels than the cultivated tomato, Lycopersicon esculentum. In contrast to the measurable levels found in the cultivated tomato, TIV1 mRNA levels in the wild tomato were reported to be undetectable (Klann et al., Plant Physiol. 103:863-870, 1993; U.S. Pat. No. 5,434,344). The sugar accumulation trait could be introduced into Lycopersicon esculentum by cross breeding and the resulting tomatoes also show altered sugar accumulation and reduced acid invertase activity (U.S. Pat. Nos. 5,434,344 and 6,072,106). These findings support the notion that a reduction in acid invertase levels results in enhanced sugar accumulation. This idea is further supported by the observation that transgenic tomatoes expressing an antisense TIV1 transgene that reduces endogenous acid invertase levels show altered sugar concentrations (Klann et al., Plant Physiol. 112:1321-1330, 1996).

In addition to the vacuolar invertase TIV1, the cell wall (apoplastic) invertase Lin5 has been implicated in sugar accumulation in tomatoes and alterations in this gene may also contribute to the differences observed between wild and cultivated tomatoes. An allele of Lin5 from the wild tomato species Lycopersicon pennellii increases total soluble solids (mainly sugars) when crossed into cultivated tomatoes (Fridman et al., PNAS 97:47184723, 2000). Comparisons of the Lycopersicon pennellii and the Lycopersicon esculentum Lin5 gene revealed sequence differences in exon 3, intron 3, and the 5′ region of exon 4. A functional polymorphism of Lin5 from wild tomato called Brix9-2-5 that affects sugar accumulation has recently been identified (Zamir et al., Science 305:1786-1789, 2004). These findings clearly establish that subtle sequence differences in the Lin5 gene can lead to important differences in sugar accumulation in the tomato.

Expression studies support the postulated role of Lin5 in sugar accumulation but also suggest a role for Lin5 in fertility and yield. Tissue specific expression studies show that Lin5 mRNA is present in green fruit, red fruits, and flowers with specific expression in gynoecia and lower levels of expression in stamen (Godt and Roitsch, Plant Physiol 115(1):273-282, 1997). In tobacco, repression of a cell wall (apoplastic) invertase in anthers results in male sterility, suggesting that sugar partitioning can affect specific tissue development. In wheat, a reduction in invertase activity precedes male sterility caused by water stress. These findings support the idea that alterations in Lin5 expression may alter fertility.

Together these data indicate that modulation of acid invertase levels can affect sugar accumulation, fertility and yield. Further, they suggest that the introduction of mutations in the vacuolar invertase TIV1 gene or the cell wall (apoplastic) invertase gene Lin5 may be used to modify sugar accumulation and affect tomato taste and processing quality. In addition, such mutations may be important for the development of sterile tomato variants, which could be important for hybrid seed production. It would be useful to have a cultivated tomato plant exhibiting these traits.

Traditional breeding methods are laborious and time consuming. In addition, undesirable characteristics are often transferred along with the desired traits when wild tomato plants are crossed with cultivated plants. Though transgenic technology can be used to modify expression of particular genes, public acceptance of genetically modified plants particularly with respect to plants used for food is low. Therefore, a cultivated tomato plant exhibiting altered sugar accumulation that was not the result of genetic engineering would be useful.

### SUMMARY OF THE INVENTION

In one aspect, this invention includes a tomato plant, fruits, seeds, plant parts and progeny thereof having an alteration in acid invertase activity caused by a non-transgenic mutation in an acid invertase gene.

In another aspect, this invention includes a tomato plant containing a mutated acid invertase gene, as well as fruit, seeds, pollen, plant parts and progeny of that plant.

In another aspect, this invention includes food and food products incorporating tomato plants having an alteration in acid invertase activity caused by a non-transgenic mutation in an acid invertase gene.

In another aspect, this invention includes a method of creating tomato plants exhibiting an alteration in acid invertase activity comprising the steps of: obtaining plant material from a desired cultivar of tomato plant; inducing at least one mutation in at least one copy of an acid invertase gene of the plant material by treating the plant material with a mutagen; culturing the mutagenized plant material to produce progeny tomato plants; analyzing progeny tomato plants to detect at least one mutation in at least one copy of an acid invertase gene; selecting progeny tomato plants that have altered acid invertase activity compared to wild type; and repeating the cycle of culturing the progeny tomato plants to produce additional progeny plants having altered acid invertase activity.

In a further aspect, this invention includes a tomato plant, fruit, seeds, pollen or plant parts created according to the method of the present invention.

### BRIEF DESCRIPTION OF THE SEQUENCE LISTING

SEQ. ID. NO.: 1 shows the genomic DNA sequence of Lycopersicon esculentum cell wall vacuolar invertase TIV1 (GenBank Accession Number Z12027).

SEQ. ID. NO.: 2 shows the coding region of the genomic DNA sequence of Lycopersicon esculentum cell wall (apoplastic) invertase Lin5 (excerpted from GenBank Accession Number AJ272306).

SEQ. ID. NOs.: 3-22 shows DNA sequences for the TIV1-specific and Lin5-specific primers of the present invention.

SEQ. ID. NO.: 23 shows the TIV1 protein sequence encoded by SEQ. ID. NO. 1 (GenBank Accession Number CAA78062).

SEQ. ID. NO.: 24 shows the Lin5 protein sequence encoded by SEQ. ID. NO. 2 (GenBank Accession Number CAB85896).

### DETAILED DESCRIPTION OF PREFERRED EMBODIMENTS

The present invention describes tomato plants exhibiting altered acid invertase enzyme activity and altered sugar profiles in the tomato fruit without the inclusion of foreign nucleic acids in the tomato plants' genomes. The present invention further describes a series of independent non-transgenic mutations in the vacuolar invertase and cell wall (apoplastic) invertase genes of tomato; tomato plants having these mutations in an acid invertase gene thereof; and a method of creating and identifying similar and/or additional mutations in acid invertase genes of tomato plants. Additionally, the present invention describes tomato plants exhibiting altered cell wall (apoplastic) invertase enzyme activity and male sterility without the inclusion of foreign nucleic acids in the plants' genomes. Furthermore, the present invention describes tomato plants exhibiting altered sugar accumulation in their tomato fruits.

In order to create and identify the acid invertase gene mutations and tomatoes of the present invention, a method known as TILLING® was utilized. See McCallum et al., Nature Biotechnology 18: 455-457, 2000; McCallum et al., Plant Physiology 123:439-442, 2000; and U.S. Pat. Nos. 5,994,075 and 20040053236, all of which are incorporated herein by reference. In the basic TILLING® methodology, plant material, such as seeds, are subjected to chemical mutagenesis, which creates a series of mutations within the genomes of the seeds' cells. The mutagenized seeds are grown into adult M1 plants and self-pollinated. DNA samples from the resulting M2 plants are pooled and are then screened for mutations in a gene of interest. Once a mutation is identified in a gene of interest, the seeds of the M2 plant carrying that mutation are grown into adult M3 plants and screened for the phenotypic characteristics associated with the gene of interest.

Any cultivar of tomato having at least one acid invertase gene with substantial homology to SEQ ID NO: 1 or SEQ ID NO: 2 may be used in the present invention. The homology between the acid invertase gene and SEQ ID NO: 1 or SEQ ID NO: 2 may be as low as 60% provided the homology in the conserved regions of the gene is higher. One of skill in the art may prefer a tomato cultivar having commercial popularity or one having specific desired characteristics in which to create the acid invertase-mutated tomato plants. Alternatively, one of skill in the art may prefer a tomato cultivar having few polymorphisms, such as an in-bred cultivar, in order to facilitate screening for mutations within an acid invertase gene.

In one embodiment of the present invention, seeds from a tomato plant were mutagenized and then grown into M1 plants. The M1 plants were then allowed to self-pollinate and seeds from the M1 plant were grown into M2 plants, which were then screened for mutations in their acid invertase genes. An advantage of screening the M2 plants is that all somatic mutations correspond to the germline mutations. One of skill in the art would understand that a variety of tomato plant materials, including but not limited to, seeds, pollen, plant tissue or plant cells, could be mutagenized in order to create the acid invertase-mutated tomato plants of the present invention. However, the type of plant material mutagenized may affect when the plant DNA is screened for mutations. For example, when pollen is subjected to mutagenesis prior to pollination of a non-mutagenized plant, the seeds resulting from that pollination are grown into M1 plants. Every cell of the M1 plants will contain mutations created in the pollen, thus these M1 plants may then be screened for acid invertase gene mutations instead of waiting until the M2 generation.

Mutagens that create primarily point mutations and short deletions, insertions, transversions, and or transitions (about 1 to about 5 nucleotides), such as chemical mutagens or radiation, may be used to create the mutations of the present invention. Mutagens conforming with the method of the present invention include, but are not limited to, ethyl methanesulfonate (EMS), methylmethane sulfonate (MMS), N-ethyl-N-nitrosurea (ENU), triethylmelamine (TEM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7, 12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9[3-(ethyl-2-chloro-ethyl)aminopropylamino] acridine dihydrochloride (ICR-170), and formaldehyde. Spontaneous mutations in an acid invertase gene that may not have been directly caused by the mutagen can also be identified using the present invention.

Any method of plant DNA preparation known to those of skill in the art may be used to prepare the tomato plant DNA for mutation screening. For example, see Chen & Ronald, Plant Molecular Biology Reporter 17:53-57, 1999; Stewart & Via, Bio Techniques 14:748-749, 1993. Additionally, several commercial kits are available, including kits from Qiagen (Valencia, Calif.) and Qbiogene (Carlsbad, Calif.).

Prepared DNA from individual tomato plants were then pooled in order to expedite screening for mutations in acid invertase genes of the entire population of plants originating from the mutagenized plant tissue. The size of the pooled group is dependent upon the sensitivity of the screening method used. Preferably, groups of four or more individuals are pooled.

After the DNA samples were pooled, the pools were subjected to acid invertase gene-specific amplification techniques, such as Polymerase Chain Reaction (PCR). For a general overview of PCR, see PCR Protocols: A Guide to Methods and Applications (Inns, M., Gelfand, D., Sninsky, J., and White, T., eds.), Academic Press, San Diego, 1990. Any primer specific to an acid invertase gene or to the sequences immediately adjacent to an acid invertase gene may be utilized to amplify an acid invertase gene within the pooled DNA sample. Preferably, the primer is designed to amplify the regions of an acid invertase gene where useful mutations are most likely to arise. It is preferable for the primer to avoid known polymorphic sites in order to ease screening for point mutations. To facilitate detection of PCR products on a gel, the PCR primer may be labeled using any conventional labeling method. In the present invention, primers were designed based upon the vacuolar invertase TIV1 gene (GenBank accession number Z12027; SEQ ID NO: 1) and the cell wall (apoplastic) invertase Lin5 gene (GenBank accession numbers AJ272306, AJ272304, and CAB85896; SEQ ID NO: 2). Exemplary primers that have proven useful in identifying useful mutations within the TIV1 gene (SEQ ID NOs: 3-16) and the Lin5 gene (SEQ ID NOs: 17-22) sequences are shown below in Table 1.

TABLE 1

| | | |
|---|---|---|
| NAME | SEQUENCE | SEQ ID NO |

| | | |
|---|---|---|
| LeTiv1L2 | CAGTGTTATGACCCCGAAAACTCCGC | 3 |
| LeTiv 1R2 | TGAGGTTGAAAATGGTAAGCCGTTCTTTG | 4 |
| LeTiv1L8 | GGCCGGGTGTAAAGCATGTGTTAAAAG | 5 |
| LeTiv1R8 | TTGAGCCTGGTTGAAGATCGACTTGCT | 6 |
| LeTiv1L4 | CAGAGGCATTTTGGGACCATTTG | 7 |
| LeTiv1R4 | GAGTCGTGCTGCTCCATTTACTGC | 8 |
| LeTiv1L6 | GCCCCCACTCAAAGTAATCCATCTTCC | 9 |
| LeTiv1R6 | TACCATTGATCAGGAACCATGGCAAAAG | 10 |
| LeTiv1L9 | GGACAAAGTCGCGCTTCAGGGAATAAT | 11 |
| LeTiv1R9 | AGTCGTGCTGCTCCATTTACTGCCTTT | 12 |
| LeTiv1L10 | TCGTTGGTCCCAGTCATTTTCTGTG | 13 |
| LeTiv1R10 | TGCAGAATAGCATCCAATCAGAATCCA | 14 |
| LeTiv1L11 | CAGGACCATTGTATCACAAGGGATGG | 15 |
| LeTiv1R11 | TGCTTTACACCCGGCCCGTTATAT | 16 |
| Lin5L2 | TGGTCAAATGAATCCGATGTATTACCTG | 17 |
| Lin5R2 | CCAAATGGTCCAAGCCCACC | 18 |
| Lin5L4 | CCATCCCGGCTAACCTATCTGATCCAT | 19 |
| Lin5R4 | TGTTGTTCAATTGGACCTTTTGCTTCC | 20 |
| Lin5L3 | CACCTGTTTTCTTCCGAGTGTTCAAG | 21 |
| Lin5R3 | ATGTTTTGCCACCAGCACCG | 22 |

The PCR amplification products may be screened for acid invertase mutations using any method that identifies nucleotide differences between wild type and mutant genes. These may include, for example, but not limited to, sequencing, denaturing high pressure liquid chromatography (dHPLC), constant denaturant capillary electrophoresis (CDCE), temperature gradient capillary electrophoresis (TGCE) (Li et al., Electrophoresis 23(10):1499-1511, 2002), or by fragmentation using enzymatic cleavage, such as used in the high throughput method described by Colbert et al., Plant Physiology 126:480-484, 2001. Preferably the PCR amplification products are incubated with an endonuclease that preferentially cleaves mismatches in heteroduplexes between wild type and mutant. Cleavage products are electrophoresed using an automated sequencing gel apparatus, and gel images are analyzed with the aid of a standard commercial image-processing program.

Mutations that reduce acid invertase function are desirable. Preferred mutations include missense and nonsense changes including mutations that prematurely truncate the translation of the acid invertase protein from messenger RNA, such as those mutations that create a stop codon within the coding region of the gene. These mutations include point mutations, insertions, repeat sequences, and modified open reading frames (ORFs). Each mutation was evaluated in order to predict its impact on protein function using the bioinformatics tools SIFT (Sorting Intolerant from Tolerant; Ng and Henikoff, Nuc Acids Res 31:3812-3814, 2003), PSSM (Position-Specific Scoring Matrix; Henikoff and Henikoff, Comput Appl Biosci 12:135-143, 1996) and PARSESNP (Taylor and Greene, Nuc Acids Res 31:3808-381, 2003). A SIFT score that is less than 0.05 and a large change in PSSM score (roughly 10 or above) indicate a mutation that is likely to have a deleterious effect on protein function. Preferable regions of interest include, but are not limited to, the coding regions in the TIV1 gene and the third and fourth exons and intervening intron of the Lin5 gene since these regions are suspected to play a regulatory role in acid invertase activity.

Once an M2 plant having a mutated acid invertase gene was identified, the mutations were analyzed to determine the affect on the expression, translation, and/or activity of the acid invertase enzyme. First, the PCR fragment containing the mutation was sequenced, using standard sequencing techniques, in order to determine the exact location of the mutation in relation to the overall acid invertase gene sequence.

If the initial assessment of the mutation in the M2 plant indicated it to be of a useful nature or in a useful position within the acid invertase gene, then further phenotypic analysis of the tomato plant containing that mutation was pursued. First, the M2 plant was backcrossed or outcrossed twice in order to eliminate background mutations. Then the M2 plant was self-pollinated in order to create a plant that was homozygous for the acid invertase mutation. However, if the acid invertase gene mutation results in complete male sterility, the M2 plant can not be self-pollinated in order to create a homozygous line. Therefore, the male sterile phenotype may be carried in a heterozygous state by crossing with pollinator lines having a wild type acid invertase gene for seed crops, or restorer lines expressing acid invertase for fruiting crops.

Physical and chemical characteristics of these homozygous acid invertase mutant plants were then assessed to determine if the mutation resulted in a useful phenotypic change in the tomato fruits. Brix values were measured to determine if there was an increase in the amount of sugar present in the fruit. Bostwick values of the fruit were measured to determine if there was a change in total solids, a measure of complex sugars.

The following mutations are exemplary of the tomato mutations created and identified in the TIV1 gene according to the present invention. One exemplary mutation in the TIV1 gene is Mutation 3689. This mutation results in a change from C to T in nucleotide 3689 of SEQ ID NO: 1 and a change from proline to leucine in amino acid 57 of the representative expressed protein SEQ ID NO: 23. Another exemplary mutation in the TIV1 gene is Mutation 6133. This mutation results in a change from A to T in nucleotide 6133 of SEQ ID NO: 1 and a change from aspartic acid to valine in amino acid 357 of the representative expressed protein SEQ ID NO: 23. Another exemplary mutation in the TIV1 gene is Mutation 6238. This mutation results in a change from C to T in nucleotide 6238 of SEQ ID NO: 1 and a change from threonine to isoleucine in amino acid 392 of the representative expressed protein SEQ ID NO: 23.

The following mutations are exemplary of the tomato mutations created and identified in the Lin5 gene according to the present invention. One exemplary mutation in the Lin5 gene is a T to A change at nucleotide 2787 of SEQ ID NO: 2. This mutation results in a change at amino acid 416 from leucine of the expressed protein [SEQ ID No: 24] to glutamine.

Another exemplary mutation, created and identified according to the present invention in the Lin5 gene, is a G to A change at nucleotide position 2284 of SEQ ID NO: 2. This mutation results in a change to a stop codon 308 from tryptophan of the expressed protein [SEQ ID NO: 24].

Another exemplary mutation in the Lin5 gene is a G to A change at nucleotide 3273 of SEQ ID NO: 2. This mutation results in a change at amino acid 515 from glutamic acid of the expressed protein [SEQ ID NO;24] to lysine.

Another exemplary mutation in the Lin5 gene is a G to A change at nucleotide 2131 of SEQ ID NO: 2. This mutation results in a change from serine at amino acid 257 in the expressed protein [SEQ ID NO: 24] to asparagine.

The following Examples are offered by way of illustration, not limitation.

### EXAMPLE 1

### Mutagenesis

Tomato seeds of cultivars Shady Lady (hybrid) and NC 84173 (an inbred line provided by Randolph G. Gardner, Director, North Carolina Agricultural Research Service, North Carolina State University) were vacuum infiltrated in H₂O (approximately 1,000 seeds/100 ml H₂O for approximately 4 minutes). The seeds were then placed on a shaker (45 rpm) in a fume hood at ambient temperature. The mutagen ethyl methanesulfonate (EMS) was added to the imbibing seeds to final concentrations ranging from about 0.1% to about 1.6% (v/v). EMS concentrations of about 0.4 to about 1.2% are preferable. Following a 24-hour incubation period, the EMS solution was replaced with fresh H₂O. The seeds were then rinsed under running water for approximately 1 hour. Finally, the mutagenized seeds were planted (96/tray) in potting soil and allowed to germinate indoors. Plants that were four to six weeks old were transferred to the field to grow to fully mature M1 plants. The mature M1 plants were allowed to self-pollinate and then seeds from the M1 plant were collected and planted to produce M2 plants.

### DNA Preparation

DNA from these M2 plants was extracted and prepared in order to identify which M2 plants carried a mutation in an acid invertase gene. The M2 plant DNA was prepared using the methods and reagents contained in the Qiagen® (Valencia, Calif.) DNeasy® 96 Plant Kit. Approximately 50 mg of frozen plant sample was placed in a sample tube with a tungsten bead, frozen in liquid nitrogen and ground 2 times for 1 minute each at 20 Hz using the Retsch® Mixer Mill MM 300. Next 400 μl of solution AP1 [Buffer AP1, solution DX and RNAse (100 mg/ml)] at 80° C. was added to the sample. The tube was sealed and shaken for 15 seconds. Following the addition of 130 μl Buffer AP2, the tube was shaken for 15 seconds. The samples were placed in a freezer at minus 20° C. for at least 1 hour. The samples were then centrifuged for 20 minutes at 5600×g. A 400 μl aliquot of supernatant was transferred to another sample tube. Following the addition of 600μl of Buffer AP3/E, this sample tube was capped and shaken for 15 seconds. A filter plate was placed on a square well block and 1 ml of the sample solution was applied to each well and the plate was sealed. The plate and block were centrifuged for 4 minutes at 5,600×g. Next, 800 μl of Buffer AW was added to each well of the filter plate, sealed and spun for 15 minutes at 5,600×g in the square well block. The filter plate was then placed on a new set of sample tubes and 80 μl of Buffer AE was applied to the filter. It was capped and incubated at room temperature for 1 minute and then spun for 2 minutes at 5,600×g. This step was repeated with an additional 80 μl Buffer AE. The filter plate was removed and the tubes containing the pooled filtrates were capped. The individual samples were then normalized to a DNA concentration of 5 to 10 ng/μl.

### TILLING®

The M2 DNA was pooled into groups of four individuals each. For pools containing four individuals, the DNA concentration for each individual within the pool was 0.25 ng/μl with a final concentration of 1 ng/μl for the entire pool. The pooled DNA samples were arrayed on microtiter plates and subjected to gene-specific PCR.

PCR amplification was performed in 15 μl volumes containing 5 ng pooled or individual DNA, 0.75X ExTaq buffer (Panvera®, Madison, Wis.), 2.6 mM MgCl₂, 0.3 mM dNTPs, 0.3 μM primers, and 0.05U Ex-Taq (Panvera®) DNA polymerase. PCR amplification was performed using an MJ Research® thermal cycler as follows: 95° C. for 2 minutes; 8 cycles of “touchdown PCR” (94° C. for 20 second, followed by annealing step starting at 70-68° C. for 30 seconds decreasing 1° C. per cycle, then a temperature ramp of 0.5° C. per second to 72° C. followed by 72° C. for 1 minute); 25-45 cycles of 94° C. for 20 seconds, 63-61° C. for 30 seconds, ramp 0.5° C./sec to 72° C., 72° C. for 1 minute; 72° C. for 8 minutes; 98° C. for 8 minutes; 80° C. for 20 second 60 cycles of 80° C. for 7 seconds −0.3° C. per cycle.

The PCR primers (MWG Biotech, Inc., High Point, N.C.) were mixed as follows:
9 μl 100 μM IRD-700 labeled left primer
1 μl 100 μM left primer
10 μl 100 μM right primer
   The IRD-700 label can be attached to either the right or left primer. Preferably, the labeled to unlabeled primer ratio is 9:1. Alternatively, Cy5.5 modified primers or IRD-800 modified primers could be used. The label was coupled to the oligonucleotide using conventional phosphoamidite chemistry.

PCR products (15 μl) were digested in 96-well plates. Next, 30 μl of a solution containing 10 mM HEPES [4-(2-hydroxyethyl)-1-piperazineethanesulfonic acid] (pH 7.5), 10 mM MgSO₄, 0.002% (w/v) Triton® X-100, 20 ng/ml of bovine serum albumin, and CEL 1 (Transgenomic®, Inc.; 1:100,000 dilution) was added with mixing on ice, and the plate was incubated at 45° C. for 15 min. The specific activity of the CEL1 was 800 units/μl, where a unit was defined by the manufacturer as the amount of enzyme required to produce 1 ng of acid-soluble material from sheared, heat denatured calf thymus DNA at pH 8.5 in one minute at 37° C. Reactions were stopped by addition of 10 μl of a 2.5 M NaCl solution with 0.5 mg/ml blue dextran and 75 mM EDTA, followed by the addition of 80 μl isopropanol. The reactions were precipitated at 80° C., spun at 4000 rpm for 30 minutes in an Eppendorf Centrifuge 5810. Pellets were resuspended in 8 μl of 33% formamide with 0.017% bromophenol blue dye, heated at 80° C. for 7 minutes and then at 95° C. for 2 minutes. Samples were transferred to a membrane comb using a comb-loading robot (MWG Biotech). The comb was inserted into a slab acrylamide gel (6.5%), electrophoresed for 10 min, and removed. Electrophoresis was continued for 4 h at 1,500-V, 40-W, and 40-mA limits at 50° C.

During electrophoresis, the gel was imaged using a LI-COR® (Lincoln, Nebr.) scanner which was set at a channel capable of detecting the IRD-700 label. The gel image showed sequence-specific pattern of background bands common to all 96 lanes. Rare events, such as mutations, create new bands that stand out above the background pattern. Plants with bands indicative of mutations of interest were evaluated by TILLING® individual members of a pool mixed with wild type DNA and then sequencing individual PCR products. Plants carrying mutations confirmed by sequencing were grown up as described above (e.g., the M2 plant was backcrossed or outcrossed twice in order to eliminate background mutations and self-pollinated in order to create a plant that was homozygous for the mutation).

### Physical and Biochemical Measurements

Tomatoes Selected for Study:

Individual tomatoes selected for study were picked from plants derived from siblings of the same cross to preserve background phenotypes as much as possible. The plants and fruit were genotyped as homozygous for the mutation, heterozygous for the mutation, or wild type. Genotyping was performed using a genetic method for determining single base pair mismatches referred to in the scientific literature as “dCAP,” see Neff et al., The Plant Journal 14:387-392, 1998. Briefly, a degenerate PCR oligonucleotide was designed to create a restriction endonuclease recognition site when the mutant base pair is present. Plants were then simply genotyped using a PCR reaction followed by a restriction enzyme digestion and then analyzed on an agarose gel. In cases where wild type siblings were not available, tomatoes from the parental cultivar were used for comparison.

Tomato Sugar Content:

Tomato sugar content was measured in Brix using a refractometer (VWR International VistaVisic hand held refractometer Catalog #12777-980). A Brix value is the percent of sucrose by weight in water. Because sugars are the main soluble solid in tomato juice, Brix is used to quantify relative amounts of sugars in tomato samples. Higher Brix values indicate higher percentages of sugars whereas lower Brix values indicate lower percentages of sugars. In general, smaller tomatoes have more concentrated sugars and higher Brix values than larger tomatoes of the same variety. Hence, average weight for each genotypic class was also recorded.

Individual tomatoes were processed for Brix measurement as follows: tomatoes were sliced, microwaved for 2 minutes to inactivate degradative enzymes, and finally pureed using a hand held blender for 30 seconds. A small amount of the puree was transferred to a microcentrifuge tube and centrifuged for 1 minute and 100 μl of the supernatant was used to determine Brix using a hand held refractometer. Brix values were found to be extremely consistent within individual samples and one reading per tomato was sufficient to establish relative Brix values for the acid invertase mutant tomatoes.

Mutations in TIV1 invertase cause a significant increase in Brix compared to wild type controls. For example, a significant increase in Brix was observed in the mutant plant line 19002 carrying the TIV1 Mutation 6238 (T392I). Because the original mutation was discovered in the homozygous state, wild type tomatoes from line 19002's parental cultivar were used as controls. Twelve control and 15 homozygous tomatoes were tested. Tomatoes from the parental cultivar had an average weight of 160 g and an average Brix of 3.9. By contrast, 19002 homozygous mutant tomatoes had an average weight of 110 g and an average Brix of 4.7. This represents an increase in Brix of 0.8% which translates to an increase in total solids of 16%. However, because the mutant tomatoes were smaller than control tomatoes, the actual gain in soluble solids due to the mutation may be slightly lower.

Mutations in Lin5 invertase caused a significant increase in Brix compared to wild type controls. For example, homozygous tomatoes from the mutant plant line 8823 carrying the Lin5 Mutation 2284 (W308*) had an average weight of 99.5 g and an average Brix of 5.4. In contrast, wild type sibling tomatoes of the mutant plant line had an average weight of 96.2 g, with an average Brix of 4.9. Thus, the mutation resulted in an increase of 0.5% of soluble sugars. The typical tomato is 95% water and 5% total solids, with 90% of the total solids represented by soluble solids and the remaining 10% represented by insoluble solids. Hence, an increase in Brix of 0.5% is a 10% increase in soluble solids. Ten wild type and 24 homozygous tomatoes were tested. Fresh tissue was unavailable for Brix measurement of tomatoes from the mutant line 12064 carrying the Lin5 Mutation 3273 (E515K); however, frozen tissue from five homozygous (n=5) and wild type control (n=5) tomatoes was thawed and Brix was measured. The average Brix of the homozygous tomatoes was 6.1 whereas the average Brix of the wild type siblings was 5.5. The Brix was higher in the mutant tomatoes than in control tomatoes; however, Brix in both groups was slightly higher than would have been observed had the tomatoes been fresh as sugars concentrate when samples dehydrate during freezer storage.

These data indicate that our mutations in TIV1 and Lin5 increase the percentage of sugar in the fruit of tomato plants carrying the mutation as reflected in increased Brix measurements.

Tomato Bostwick Consistency:

Another means of measuring the increase in complex sugars in the tomato is as a function of the increase in total solids as determined by the thickness of pureed tomato tissue. A Bostwick Consistometer, a simple mechanical device for measuring total solids, is composed of a spring-gated compartment and ramp at a slight incline to facilitate tomato puree flow. Puree is poured into the compartment and the gate is released to allow the puree to flow down the ramp. The Bostwick value is the distance (in cm) that the tomato puree travels in a defined period of time (Barrett et al., Critical Reviews in Food Sciences and Nutrition 38:173-258, 1998).

Individual tomatoes were processed for Bostwick consistency as follows: tomatoes were sliced, microwaved for 2 minutes to inactivate degradative enzymes, and finally pureed using a hand held blender for 30 seconds. The tomato puree was cooled to room temperature and 50 mls of puree from each tomato sample were allowed to flow for 30 seconds down the Bostwick ramp. The distance traveled was recorded in centimeters.

Mutations in TIV1 invertase caused a significant decrease in Bostwick consistency compared to wild type controls. For example, puree made from homozygous tomatoes of the mutant plant line 12401 carrying the TIV1 Mutation 3689 (P57L) flowed only 6.6 cm whereas puree made from wild type sibling tomatoes flowed an average of 7.9 cm. Twenty-three wild type and 92 homozygous tomatoes were tested.

Mutations in Lin5 invertase also caused a significant decrease in Bostwick consistency compared to wild type controls. For example, puree made from homozygous mutant tomatoes of the mutant plant line 8823 carrying the Lin5 Mutation 2284 (W308*) flowed an average of 3.6 cm whereas puree made from wild type sibling tomatoes flowed an average of 7.6 cm. Four wild type and four homozygous tomatoes were tested.

These data indicate that our mutations in TIV1 and Lin5 increase total solids in the fruit of tomato plants carrying the mutations as reflected by a decrease in Bostwick consistency compared to control tomato fruit.

Tomato Taste Test:

Fifteen people were asked to taste fresh tomato samples and to judge blindly which sample was sweeter. The samples included homozygous tomatoes from the mutant line 12064 carrying the Lin5 Mutation 3273 (E515K) and wild type sibling controls. The homozygous mutant tomatoes were judged sweeter by 54% of the people whereas wild type sibling control tomatoes were considered sweeter by only 31 % of people. Fifteen percent of people were unable to discern a difference between the groups. In other words, 1.7 times as many people found the Lin5 mutant tomatoes to be sweeter than the control tomatoes. Brix measurements were performed later on frozen tomato samples from the same plants that were used in the taste test. The results confirmed that tomatoes from the homozygous mutant line were perceived to be sweeter by judges who were blind to the knowledge that the mutant tomatoes had a slightly higher Brix than the wild type control tomatoes.

### Identification and Evaluation of TIV1 Mutation 3689

DNA from a tomato plant originating from seeds of cultivar Shady Lady that were incubated in 0.8% EMS, was amplified using primers LeTiv1L2 and LeTiv1R2 (SEQ ID NOs: 3 and 4). The PCR amplification products were then incubated with CEL 1 and electrophoresed. The electrophoresis gel image showed a fragment that stood out above the background pattern for the PCR amplification products. Therefore, it was likely that this fragment contained a heteroduplex created by a mutation in the TIV1 sequence. Sequence analysis of this fragment showed the mutation was a C to T change at nucleotide 3689 of SEQ ID NO: 1. This mutation was associated with a change from proline to leucine at amino acid 57 of the TIV1 polypeptide [SEQ ID NO: 23].

### Identification and Evaluation of TIV1 Mutation 6133

DNA from a tomato plant originating from seeds of cultivar Shady Lady that were incubated in 0.8% EMS, was amplified using primers LeTiv1L8 and LeTiv1R8 (SEQ ID NOs: 5 and 6). The PCR amplification products were then incubated with CEL 1 and electrophoresed. The electrophoresis gel image showed a fragment that stood out above the background pattern for the PCR amplification products. Therefore, it was likely that this fragment contained a heteroduplex created by a mutation in the TIV1 sequence. Sequence analysis of this fragment showed the mutation was a A to T change at nucleotide 6133 of SEQ ID NO: 1. This mutation was associated with a change from aspartic acid to valine at amino acid 357 of the TIV1 polypeptide [SEQ ID NO: 23].

### Identification and Evaluation of TIV1 Mutation 6238

DNA from a tomato plant originating from seeds of cultivar Shady Lady that were incubated in 0.8% EMS, was amplified using primers LeTiv1L8 and LeTiv1R8 (SEQ ID NOs: 5 and 6). The PCR amplification products were then incubated with CEL 1 and electrophoresed. The electrophoresis gel image showed a fragment that stood out above the background pattern for the PCR amplification products. Therefore, it was likely that this fragment contained a heteroduplex created by a mutation in the TIV1 sequence. Sequence analysis of this fragment showed the mutation was a C to T change at nucleotide 6238 of SEQ ID NO: 1. This mutation was associated with a change from threonine to isoleucine at amino acid 392 of the TIV1 polypeptide [SEQ ID NO: 23].

### Identification and Evaluation of Lin5 Mutation 2787

DNA from a tomato plant originating from seeds of cultivar Shady Lady that were incubated in 0.8% EMS, was amplified using primers Lin5L2 and Lin5R2 (SEQ ID NOs: 17 and 18). The PCR amplification products were then incubated with CEL 1 and electrophoresed. The electrophoresis gel image showed a fragment that stood out above the background pattern for the PCR amplification products. Therefore, it was likely that this fragment contained a heteroduplex created by a mutation in the Lin5 gene. Sequence analysis of this fragment showed the mutation was a T to A change at nucleotide 2787 of SEQ ID NO: 2. This mutation correlates with a change from leucine at amino acid 416 of the Lin5 polypeptide [SEQ ID NO: 24] to glutamine.

### Identification and Evaluation of Lin5 Mutation 2284

Tomato plant originating from seeds of cultivar Shady Lady that were incubated in 0.8% EMS, was screened with primers Lin5L4 and Lin5R4 (SEQ ID NOs: 19 and 20). The PCR amplification products were then incubated with CEL 1 and electrophoresed. The electrophoresis gel image showed a fragment that stood out above the background pattern for the PCR amplification products. Therefore, it was likely that this fragment contained a heteroduplex created by a mutation in the Lin5 gene. Sequence analysis of this fragment showed the mutation was a G to A change at nucleotide 2284 of SEQ ID NO: 2. This mutation correlates with an amino acid change from tryptophan at 308 of the Lin5 polypeptide [SEQ ID NO: 24] to a stop codon.

### Identification and Evaluation of Lin5 Mutation 3273

DNA from a tomato plant originating from seeds of cultivar Shady Lady that were incubated in 0.6% EMS, was amplified using primers Lin5L3 and Lin5R3 (SEQ ID NOs: 21 and 22). The PCR amplification products were then incubated with CEL 1 and electrophoresed. The electrophoresis gel image showed a fragment that stood out above the background pattern for the PCR amplification products. Sequence analysis of this fragment showed the mutation was a G to A change at nucleotide 3273 of SEQ ID NO: 2. This mutation correlates with a change from glutamic acid at amino acid 515 of the Lin5 polypeptide [SEQ ID NO: 24] to lysine.

### Identification and Evaluation of Lin5 Mutation 2131

DNA from a tomato plant originating from seeds of cultivar Shady Lady that were incubated in 0.8% EMS, was amplified using primers Lin5L4 and Lin5R4 (SEQ ID NOs: 19 and 20). The PCR amplification products were then incubated with CEL 1 and electrophoresed. The electrophoresis gel image showed a fragment that stood out above the background pattern for the PCR amplification products. Therefore, it was likely that this fragment contained a heteroduplex created by a mutation in the Lin5 gene. Sequence analysis of this fragment showed the mutation was a G to A change at nucleotide 2131 of SEQ ID NO: 2. This mutation correlates with a change from serine at amino acid 257 of the Lin5 polypeptide [SEQ ID NO: 24] to asparagine.

### Deposit Information

A representative deposit of Lycopersicon esculentum seeds of the cultivar Shady Lady containing the Lin5 Mutation 2787 was deposited with the American Type Culture Collection, 10801 University Blvd., Mannassas, Va. 20110-2209, on Nov. 14, 2003 and given Accession No. 19758 and Patent Deposit Designation PTA-563 1. A representative deposit of Lycopersicon esculentum seeds of the cultivar Shady Lady containing the Lin5 Mutation 3273 was deposited with the American Type Culture Collection, 10801 University Blvd., Mannassas, Va. 20110-2209, on Nov. 14, 2003 and given Accession No. 12064 and Patent Deposit Designation PTA-5629. A representative deposit of Lycopersicon esculentum seeds of the cultivar Shady Lady containing the Lin5 Mutation 2131 was deposited with the American Type Culture Collection, 10801 University Blvd., Mannassas, Va. 20110-2209, on Nov. 14, 2003 and given Accession No. 19023 and Patent Deposit Designation PTA-5630. Additionally, if deemed necessary by the Commissioner of Patents and Trademarks or any persons acting on his behalf, Applicants will make a deposit of at least 2500 seeds for each of the tomato varieties containing an exemplary mutation described in this application with the American Type Culture Collection (ATCC). The seeds deposited with the ATCC will be taken from the deposit maintained by Anawah, Inc., 1102 Columbia Street, Suite 600, Seattle, Wash., 98104, since prior to the filing date of this application. Access to this deposit will be available during the pendency of the application to the Commissioner of Patents and Trademarks and persons determined by the Commissioner to be entitled thereto upon request. Upon allowance of any claims in the application and if designated by the Commissioner of Patents and Trademarks as a condition for allowance of those claims, Applicants will make the deposit available to the public pursuant to 37 CFR 1.808. All deposits related to this application will be maintained in the ATCC depository, which is a public depository, for a period of 30 years, or 5 years after the most recent request, or for the enforceable life of the patent, whichever is longer, and will be replaced if it becomes nonviable during that period. Additionally, Applicants have or will satisfy all requirements of 37 CFR Sections 1.801-1.809, including providing an indication of the viability of the sample upon deposit. Applicants have no authority to waive any restrictions imposed by law o the transfer of biological material or its transportation in commerce. Applicants do not waive any infringement of their rights granted under this patent or under the Plant Variety Protection Act (7 USC 2321 et seq.).

The above examples are provided to illustrate the invention but not limit its scope. Other variants of the invention will be readily apparent to one of ordinary skill in the art and are encompassed by the appended claims and all their equivalents. All publications, patents, and patent applications cited herein are hereby incorporated by reference.

1. A method of creating a tomato plant exhibiting an altered acid invertase activity compared to wild type tomato plants, comprising the steps of:
a. obtaining plant material from a parent tomato plant;
b. inducing at least one mutation in at least one copy of an acid invertase gene of the plant material by treating the plant material with a mutagen to create mutagenized plant material;
c. culturing the mutagenized plant material to produce progeny tomato plants;
d. analyzing progeny tomato plants to detect at least one mutation in at least one copy of an acid invertase gene;
e. selecting progeny tomato plants that have altered acid invertase enzyme activity compared to a wild type tomato plant; and
f. repeating the cycle of culturing the progeny tomato plants to produce additional progeny tomato plants having altered acid invertase enzyme activity.

2. The method of claim 1 wherein the acid invertase gene is a Lin5 apoplastic invertase gene.

3. The method of claim 2 where the progeny tomato plant are analyzed by
a. isolating genomic DNA from the progeny tomato plants; and
b. amplifying segments of the Lin5 apoplastic invertase gene in the isolated genomic DNA using primers specific to the Lin5 apoplastic invertase gene or to the DNA sequences adjacent to the Lin5 apoplastic invertase gene.

4. The method of claim 3 where at least one primer has a sequence substantially homologous to a sequence in the group consisting of SEQ. ID. NOs. 17 through 22.

5. The method of claim 1 wherein the acid invertase gene is a TIV1 vacuolar invertase gene.

6. The method of claim 5 where the progeny tomato plant are analyzed by
a. isolating genomic DNA from the progeny tomato plants; and
b. amplifying segments of the TIV1 vacuolar invertase gene in the isolated genomic DNA using primers specific to the TIV1 vacuolar invertase gene or to the DNA sequences adjacent to the TIV1 vacuolar invertase gene.

7. The method of claim 6 where at least one primer has a sequence substantially homologous to a sequence in the group consisting of SEQ. ID. NOs. 3 through 16.

8. The method of claim 1 wherein the plant material is selected from the group consisting of seeds, pollen, plant cells, or plant tissue.

9. The method of claim 1 wherein the mutagen is ethyl methanesulfonate.

10. The method of claim 9 wherein the concentration of ethyl methanesulfonate used is from 0.4 to about 1.2%.

11. The method of claim 1 wherein the mutation detected in step d is evaluated to determine the mutation's likelihood of having a deleterious effect on acid invertase enzyme activity.

12. The method of claim 11 where in the mutation is evaluated using a bioinformatics tool selected from the group consisting of SIFT, PSSM and PARSESNP.

13. Tomato fruit, seeds, pollen, plant parts or progeny of the tomato plant created according to the method of claim 1.

14. Food and food products incorporating a tomato fruit created according to the method of claim 1.

15. A tomato plant, tomato fruit, seeds, plant parts, and progeny thereof having altered acid invertase activity compared to a wild type tomato plant wherein the altered acid invertase activity is caused by a non-transgenic mutation in an acid invertase gene.

16. A tomato plant, tomato fruit, seeds, plant parts, and progeny thereof of claim 15 wherein the acid invertase gene is a Lin5 apoplastic invertase and the non-transgenic mutation is in a Lin5 apoplastic invertase gene.

17. A tomato plant, tomato fruit, seeds, plant parts, and progeny thereof of claim 15 wherein the acid invertase is a TIV1 vacuolar invertase and the non-transgenic mutation is in a TIV1 vacuolar invertase gene.

18. A tomato fruit having an increased sugar content when ripe compared to a wild type tomato fruit due to an alteration in acid invertase activity caused by a non-transgenic mutation in an acid invertase gene.

19. A tomato fruit of claim 18 wherein the acid invertase gene is a Lin5 apoplastic invertase and the non-transgenic mutation is in a Lin5 apoplastic invertase gene.

20. A tomato plant, seeds, plant parts, pollen and progeny thereof capable of producing the tomato fruit of claim 19.

21. A tomato having an increased sugar content obtainable by crossing a plant or pollen of claim 20 with another plant showing a desired phenotype with respect to sugar content.

22. Food and food products incorporating the tomato fruit of claim 19.

23. A tomato fruit of claim 18 wherein the acid invertase is a TIV1 vacuolar invertase and the non-transgenic mutation is in a TIV1 vacuolar invertase gene.

24. A tomato plant, seeds, plant parts, pollen and progeny thereof capable of producing the tomato fruit of claim 23.

25. A tomato having an increased sugar content obtainable by crossing a plant or pollen of claim 24 with another plant showing a desired phenotype with respect to sugar content.

26. Food and food products incorporating the tomato fruit of claim 23.

27. A tomato plant, tomatoes, seeds, plant parts, and progeny thereof having an altered apoplastic invertase activity caused by a non-transgenic mutation in the Lin5 gene.

28. The tomato plant, fruit, seeds, plant parts and progeny of claim 27 wherein the non-transgenic mutation is a point mutation.

29. The tomato plant, tomatoes, seeds, plant parts and progeny of claim 27 wherein the non-transgenic mutation creates a change in at least amino acid 416 of the invertase enzyme expressed from the Lin5 gene.

30. The tomato plant, tomatoes, seeds, plant parts and progeny of claim 27 wherein the non-transgenic mutation creates a change in at least amino acid 308 of the invertase enzyme expressed from the Lin5 gene.

31. The tomato plant, tomatoes, seeds, plant parts and progeny of claim 27 wherein the non-transgenic mutation creates a change in at least amino acid 515 of the invertase enzyme expressed from the Lin5 gene.

32. The tomato plant, tomatoes, seeds, plant parts and progeny of claim 27 wherein the non-transgenic mutation creates a change in at least amino acid 257 of the invertase enzyme expressed from the Lin5 gene.

33. The tomato plant, tomatoes, seeds, plant parts and progeny of claim 27 wherein the non-transgenic mutation creates a change in at least amino acid 350 of the invertase enzyme expressed from the Lin5 gene.

34. Food and food products incorporating a tomato of claim 27.

35. Pollen from the tomato plant of claim 27.

36. A endogenous Lin5 apoplastic invertase gene having substantial homology to SEQ. I.D. No. 2 and having at least one mutation in the third or fourth intron or intervening exon of the endogenous apoplastic invertase gene.

37. An endogenous Lin5 apoplastic invertase gene having at least a point mutation at around nucleotide 2787 of SEQ ID NO:2.

38. A plant containing the mutated Lin5 apoplastic invertase gene of claim 37.

39. Fruit, seeds, pollen, plant parts and progeny of the plant of claim 38.

40. Food and food products incorporating the fruit of the plant of claim 38.

41. A protein expressed from the mutated Lin5 apoplastic invertase gene of claim 37, having a glutamine at amino acid 416.

42. An endogenous Lin5 apoplastic invertase gene having at least a point mutation at around nucleotide 2284 of SEQ ID NO:2.

43. A plant containing the mutated Lin5 apoplastic invertase gene of claim 42.

44. Fruit, seeds, pollen, plant parts and progeny of the plant of claim 43.

45. Food and food products incorporating the fruit of the plant of claim 43.

46. A mutated endogenous Lin5 apoplastic invertase gene having at least a point mutation at around nucleotide 3273 of SEQ ID NO:2.

47. A plant containing the mutated Lin5 apoplastic invertase gene of claim 46.

48. Fruit, seeds, pollen, plant parts and progeny of the plant of claim 47.

49. Food and food products incorporating the fruit of the plant of claim 47.

50. A protein expressed from the mutated Lin5 apoplastic invertase gene of claim 46, having a lysine at amino acid 515.

51. A mutated endogenous Lin5 apoplastic invertase gene having at least a point mutation at around nucleotide 2131 of SEQ ID NO:2.

52. A plant containing the mutated Lin5 apoplastic invertase gene of claim 51.

53. Fruit, seeds, pollen, plant parts and progeny of the plant of claim 52.

54. Food and food products incorporating the fruit of the plant of claim 52.

55. A protein expressed from the mutated Lin5 apoplastic invertase gene of claim 51, having a aspargine at amino acid 257.

56. A mutated endogenous Lin5 apoplastic invertase gene having at least a point mutation at around nucleotide 2410 of SEQ ID NO:2.

57. A plant containing the mutated Lin5 apoplastic invertase gene of claim 56.

58. Fruit, seeds, pollen, plant parts and progeny of the plant of claim 57.

59. Food and food products incorporating the fruit of the plant of claim 57.

60. A protein expressed from the mutated Lin5 apoplastic invertase gene of claim 56, having a methionine at amino acid 350.

61. A tomato plant, tomatoes, seeds, plant parts and progeny thereof exhibiting male sterility caused by a non-transgenic mutation in the Lin5 apoplastic invertase gene.

62. An endogenous TIV1 vacuolar invertase gene having substantial homology to SEQ. I.D. No. 1 and having a non-transgenic mutation within the endogenous TIV1 vacuolar invertase gene.

63. The endogenous TIV1 vacuolar invertase gene of claim 62 wherein the non-transgenic mutation occurs around nucleotide 3689.

64. A tomato plant containing the endogenous TIV1 vacuolar invertase gene of claim 63.

65. Fruit, seeds, pollen, plant parts, and progeny of the tomato plant of claim 64.

66. Food and food products incorporating a tomato fruit of claim 65.

67. The endogenous TIV1 vacuolar invertase gene of claim 62 wherein the non-transgenic mutation creates a change in at least amino acid 57 of the vacuolar invertase enzyme expressed from the TIV1 vacuolar invertase gene.

68. An invertase enzyme expressed from the endogenous TIV1 vacuolar invertase gene of claim 67.

69. The vacuolar invertase enzyme of claim 68 having a leucine at amino acid 57.

70. The endogenous TIV1 vacuolar invertase gene of claim 62 wherein the non-transgenic mutation occurs around nucleotide 6133.

71. A tomato plant containing the endogenous TIV1 vacuolar invertase gene of claim 70.

72. Fruit, seeds, pollen, plant parts, and progeny of the tomato plant of claim 71.

73. Food and food products incorporating a tomato fruit of claim 72.

74. The endogenous TIV1 vacuolar invertase gene of claim 62 wherein the non-transgenic mutation creates a change in at least amino acid 357 of the vacuolar invertase enzyme expressed from the TIV1 vacuolar invertase gene.

75. An invertase enzyme expressed from the endogenous TIV1 vacuolar invertase gene of claim 74.

76. The invertase enzyme of claim 75 having a valine at amino acid 357.

77. The endogenous TIV1 vacuolar invertase gene of claim 62 wherein the non-transgenic mutation occurs around nucleotide 6238.

78. A tomato plant containing the endogenous TIV1 vacuolar invertase gene of claim 77.

79. Tomato fruits, seeds, pollen, plant parts, and progeny of the tomato plant of claim 78.

80. Food and food products incorporating a tomato fruit of claim 79.

81. The endogenous TIV1 vacuolar invertase gene of claim 62 wherein the non-transgenic mutation creates a change in at least amino acid 392 of the vacuolar invertase enzyme expressed from the TIV1 vacuolar invertase gene.

82. An invertase enzyme expressed from the endogenous TIV1 vacuolar invertase gene of claim 81.

83. The invertase enzyme of claim 82 having an isoleucine at amino acid 392.
